(19) United States
(12) Patent Application Publication
(10) Pub. No.: US 2017/0224309 A1
(43) Pub. Date: Aug. 10, 2017

(54) ULTRASONIC DIAGNOSTIC DEVICE

(71) Applicant: Toshiba Medical Systems Corporation, Otawara-shi (JP)

(72) Inventor: Tomohisa IMAMURA, Kawasaki (JP)

(73) Assignee: Toshiba Medical Systems Corporation, Otawara-shi (JP)

(21) Appl. No.: 15/427,569

(22) Filed: Feb. 8, 2017

(30) Foreign Application Priority Data
Feb. 10, 2016 (JP) 2016-023899
Aug. 31, 2016 (JP) 2016-170257

(51) Int. Cl. A61B 8/08; A61B 8/06; A61B 8/00

(52) U.S. Cl. CPC A61B 8/488; A61B 8/5207; A61B 8/483; A61B 8/461; A61B 8/06; A61B 8/56

(57) ABSTRACT

An ultrasonic diagnostic device according to an embodiment includes transmission and reception circuitry and processing circuitry. The transmission and reception circuitry repeatedly executes first scanning in which a subject is scanned along a plurality of scanning lines. The processing circuitry controls generating a color Doppler image based on reflected wave data collected through the first scanning. The processing circuitry controls obtaining a wait time of the first scanning based on a repetition period of the first scanning and a required time of the first scanning. The transmission and reception circuitry executes second scanning different from the first scanning during the wait time obtained by the processing circuitry.

### CROSS-REFERENCE TO RELATED APPLICATIONS

This application is based upon and claims the benefit of priority from Japanese Patent Application No. 2016-023899, filed on Feb. 10, 2016; and Japanese Patent Application No. 2016-170257, filed on Aug. 31, 2016, the entire contents of all of which are incorporated herein by reference.

### FIELD

Embodiments described herein generally relate to an ultrasonic diagnostic device.

### BACKGROUND

The conventional ultrasonic diagnostic device has a function to perform, by a Doppler technique based on the Doppler effect, generation of bloodstream information from reflected wave of ultrasonic rave and display of the bloodstream information. Examples of the bloodstream information displayed by the ultrasonic diagnostic device include a color Doppler image and a Doppler waveform (Doppler spectrum).

The color Doppler image is an ultrasonic image captured by a color flow mapping (CFM) method. The CFM method performs transmission and reception of ultrasonic wave on a plurality of scanning lines in a region (two-dimensional or three-dimensional region) including an observation site and a diagnosis site. The CFM method extracts bloodstream component data from reflected wave data through a moving target indicator (MTI) filter by removing a frequency component (clutter component) attributable to motion of tissue. Then, the CFM method performs a frequency analysis of the bloodstream component data by an autocorrelation method so as to calculate the speed of bloodstream, the dispersion of bloodstream, and the power of bloodstream. The color Doppler image is an ultrasonic image in which distribution of this calculation result is two-dimensionally displayed in color.

### BRIEF DESCRIPTION OF THE DRAWINGS

FIG. 1 is a block diagram of an exemplary configuration of an ultrasonic diagnostic device according to a first embodiment;

FIG. 2 is a diagram illustrating an exemplary display image displayed by processing at processing circuitry according to the first embodiment;

FIG. 3 is a diagram for describing a region scanned by transmission and reception circuitry according to the first embodiment;

FIG. 4 is a diagram for describing the region scanned by the transmission and reception circuitry according to the first embodiment;

FIG. 5 is a diagram for describing an order of scanning by the transmission and reception circuitry according to the first embodiment;

FIG. 6 is a diagram for describing processing of calculating a wait time by a calculation function according to the first embodiment;

FIG. 7 is a flowchart of the procedure of processing at the processing circuitry according to the first embodiment;

FIG. 8 is a diagram for describing a region scanned by a transmission and reception circuitry according to a first modification of the first embodiment;

FIG. 9 is a diagram for describing processing of calculating a wait time by a calculation function according to the first modification of the first embodiment;

FIG. 10 is a diagram for describing a region scanned by transmission and reception circuitry according to a second modification of the first embodiment;

FIG. 11 is a block diagram of an exemplary configuration of an ultrasonic diagnostic device according to a second embodiment;

FIGS. 12A and 12B are diagrams for describing processing at a control function according to the second embodiment;

FIG. 13 is a block diagram of an exemplary configuration of an ultrasonic diagnostic device according to a modification of the second embodiment; and

FIG. 14 is a diagram for describing processing at a control function according to the modification of the second embodiment.

### DETAILED DESCRIPTION

An ultrasonic diagnostic device according to an embodiment includes transmission and reception circuitry and processing circuitry. The transmission and reception circuitry repeatedly executes a first scanning in which a subject is scanned along a plurality of scanning lines. The the processing circuitry controls generating a color Doppler image on the basis of reflected wave data collected through the first scanning. The processing circuitry controls obtaining a wait time of the first scanning on the basis of a repetition period of the first scanning and a required time of first scanning. The transmission and reception circuitry executes a second scanning different from the first scanning during the wait time obtained by the processing circuitry.

An ultrasonic diagnostic device according to embodiments will be described below with reference to the accompanying drawings.

### First Embodiment

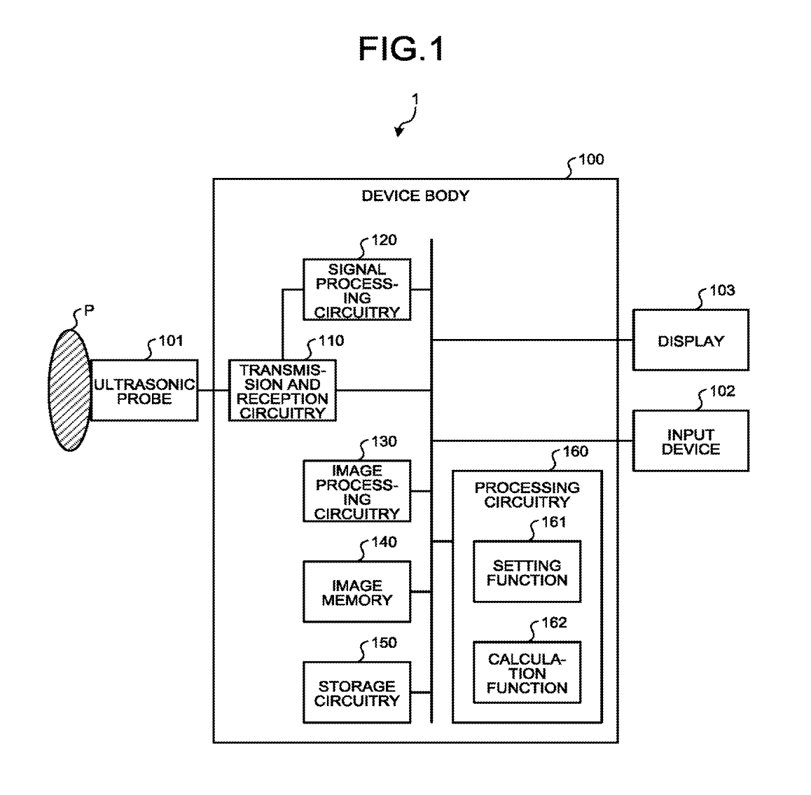

FIG. 1 is a block diagram of an exemplary configuration of an ultrasonic diagnostic device 1 according to a first embodiment. As illustrated in FIG. 1, the ultrasonic diagnostic device 1 according to the first embodiment includes an ultrasonic probe 101, an input device 102, a display 103, and a device body 100. The ultrasonic probe 101, the input device 102, and the display 103 are connected with the device body 100 to perform communication therebetween. subject P is not included in the configuration of the ultrasonic diagnostic device 1.

The ultrasonic probe 101 performs transmission and reception of ultrasonic wave. For example, the ultrasonic probe 101 includes a plurality of piezoelectric transducer elements (also called transducer elements). These piezoelectric transducer elements generate an ultrasonic wave on the basis of a drive signal supplied from a transmission and reception circuitry 110 included in the device body 100 to be described later. The piezoelectric transducer elements included in the ultrasonic probe 101 receives a reflected wave from the subject P and converts the reflected wave into an electric signal. The ultrasonic probe 101 includes, for example, a matching layer provided to the piezoelectric transducer elements, and a backing material that prevents ultrasonic wave from traveling beyond the piezoelectric transducer elements. The ultrasonic probe 101 is detachably connected with the device body 100.

When the ultrasonic probe 101 transmits the ultrasonic wave to the subject P, the transmitted ultrasonic wave is sequentially reflected at discontinuous surfaces of acoustic impedance in body tissue of the subject P, and received as a reflected wave signal by the piezoelectric transducer elements included in the ultrasonic probe 101. The amplitude of the reflected wave signal thus received depends on a difference in acoustic impedance between the discontinuous surfer at which the ultrasonic wave is reflected. When a transmitted ultrasonic pulse is reflected at moving bloodstream and a surface such as a cardiac wall, a reflected wave signal is provided with a frequency shift through the Doppler effect in accordance with a speed component of a moving body in an ultrasonic wave transmission direction.

The ultrasonic probe 101 according to the first embodiment may be a 1D array probe that two-dimensionally scans the subject P, or a mechanical 4D probe or a 2D array probe that three-dimensionally scans the subject P.

The input device 102 corresponds to a device such as a mouse, a keyboard, a button, a panel switch, a touch command screen, a foot switch, a track ball, or a joystick. The input device 102 receives various setting requests from an operator of the ultrasonic diagnostic device 1, and forwards the various setting requests thus received to the device body 100.

The display 103 displays thereon, for example, a graphical user interface (GUI) that allows the operator of the ultrasonic diagnostic device 1 to input various setting requests through the input device 102, and ultrasonic image data generated at the device body 100.

The device body 100 is a device configured to generate ultrasonic image data on the basis of a reflected wave signal received by the ultrasonic probe 101. Ultrasonic image data generated by the device body 100 illustrated in FIG. 1 may be two-dimensional ultrasonic image data generated on the basis of a two-dimensional reflected wave signal, or three-dimensional ultrasonic image data generated on the basis of a three-dimensional reflected wave signal.

As exemplarily illustrated in FIG. 1, the device body 100 includes the transmission and reception circuitry 110, signal processing circuitry 120, image processing circuitry 130, an image memory 140, storage circuitry 150, and processing circuitry 160. The transmission and reception circuitry 110, the signal processing circuitry 120, the image processing circuitry 130, the image memory 140, the storage circuitry 150, and the processing circuitry 160 are connected with each other to perform communication therebetween.

The transmission and reception circuitry 110 controls ultrasonic wave transmission and reception performed by the ultrasonic probe 101 on the basis of an instruction from the processing circuitry 160 to be described later. The transmission and reception circuitry 110 includes, for example, a pulse generator, a transmission delay circuit, and a pulser, and supplies a drive signal to the ultrasonic probe 101. The pulse generator generates a rate pulse for forming ultrasonic wave to be transmitted. The transmission delay circuit focuses ultrasonic wave generated by the ultrasonic probe 101 into a beam, and provides each rate pulse generated by the pulse generator with a delay time for each piezoelectric transducer element, which is necessary for determining transmission directionality. The pulser applies a drive signal (drive pulse) to the ultrasonic probe 101 at a timing based on the rate pulse. In other words, the transmission delay circuit optionally adjusts a transmission direction of the ultrasonic wave transmitted from a surface of the piezoelectric transducer element by providing each rate pulse with a different delay time.

The transmission and reception circuitry 110 has a function to instantaneously change a transmission frequency, a transmission drive voltage, and the like to execute a predetermined scanning sequence on the basis of an instruction from the processing circuitry 160 to be described later. In particular, the change of the transmission drive voltage is achieved by a linear-amplifier oscillation circuit capable of instantaneously switching the value of the transmission drive voltage, or a mechanism for electrically switching a plurality of power units.

The transmission and reception circuitry 110 includes, for example an amplifier circuit, an analog/digital (A/D) converter, an adder, and a phase detection circuit, and generates reflected wave data by performing various kinds of processing on a reflected wave signal received by the ultrasonic probe 101. The amplifier circuit performs gain correction processing by amplifying the reflected wave signal for each channel. The A/D converter performs A/D conversion of the reflected wave signal provided with the gain correction, and provides digital data with a delay time necessary for determining reception directionality. The adder performs addition processing of the reflected wave signal processed by the A/D converter. The addition processing by the adder enhances a reflection component in a direction in accordance with the reception directionality of the reflected wave signal. The phase detection circuit converts an output signal from the adder into an in-phase signal (I signal) and a quadrature-phase signal (Q signal) of a baseband band. Then, the phase detection circuit outputs the I signal and the Q signal (IQ signal) to the signal processing circuitry 120 at a later stage. Data before the processing at the phase detection circuit is also called an RD signal. In the following, the IQ signal and the RD signal generated on the basis of the reflected wave of the ultrasonic wave are collectively referred to as “reflected wave data”.

The transmission and reception circuitry 110 can generate reflected wave data of a plurality of scanning lines from a plurality of reflected wave signals obtained by a single ultrasonic wave transmission. In other words, the transmission and reception circuitry 110 is capable of performing parallel simultaneous reception processing. The first embodiment is applicable to a case in which the transmission and reception circuitry 110 cannot execute the parallel simultaneous reception processing.

The signal processing circuitry 120 is a signal processing unit configured to perform various kinds of signal processing on the reflected wave data generated from the reflected wave signal by the transmission and reception circuitry 110. The signal processing circuitry 120 generates data (B mode data) on a signal intensity at multiple points represented by the magnitude of a luminance, by performing, for example, logarithmic amplification, envelope detection processing, logarithmic compression on the reflected wave data (IQ signal) read from a buffer. The signal processing circuitry 120 may generate the E mode data by performing synthesis (addition, subtraction, or their combination) of reflected wave data on an identical reception scanning line, which is obtained through a plurality of times of ultrasonic wave transmission under scanning conditions different from each other. The scanning conditions include at least one of the phase and steering angle of transmitted ultrasonic wave, a group (transmission opening) of piezoelectric transducer elements used to transmit ultrasonic wave, and a group (reception opening) of piezoelectric transducer elements used to receive ultrasonic wave, a transmission frequency, and a reception frequency. The above-described synthesis can be executed whether or not the reflected wave data has phase information. The transmitted ultrasonic wave may have a plurality of central frequencies.

The signal processing circuitry 120 generates data (Doppler data) obtained by extracting motion information on the basis of the Doppler effect of a moving body in a scanning range by performing frequency analysis of the reflected wave data. Specifically, the signal processing circuitry 120 generates, as the motion information on the moving body, Doppler data obtained by estimating, for example, an average speed, an average dispersion value, and an average power value for each of a plurality of sample points. The moving body is, for example, tissue such as bloodstream and a cardiac wall, or contrast agent. The signal processing circuitry 120 according to the present embodiment generates, as the motion information (bloodstream information) on bloodstream, Doppler data obtained by estimating, for example, the average speed of bloodstream, the average dispersion value of bloodstream, and the average power value of bloodstream for each of a plurality of sample points.

By using the above-described function of the signal processing circuitry 120, the ultrasonic diagnostic device 1 according to the present embodiment can execute a color Doppler technique also called a color flow mapping (CFM) method. In the CFM method, transmission and reception of ultrasonic wave is performed a plurality of times on a plurality of scanning lines. A data string of reflected wave signals (reflected wave data) from an identical position obtained by this ultrasonic wave transmission and reception is called a packet. The packet has a size corresponding the number of times of ultrasonic wave transmission and reception performed in an identical direction to obtain the bloodstream information of one frame.

Then, the CFM method applies a moving target indicator (MTI) filter on the data string from an identical position so as to reduce a signal (clutter signal) attributable to tissue at rest or slowly moving tissue, and extract a signal attributable to bloodstream. The CFM method estimates, from this bloodstream signal, the bloodstream information such as the speed of bloodstream, the dispersion of bloodstream, and the power of bloodstream. The image processing circuitry 130 to be described later generates, for example, ultrasonic image data (color Doppler image data) in which distribution of results of estimation is two-dimensionally displayed in color. Then, the display 103 displays the color Doppler image data. As the size of the packet increases, the performance of reduction of the clutter signal improves, but a frame rate decreases.

The signal processing circuitry 120 outputs a data string from which a clutter component is reduced and a bloodstream signal attributable bloodstream is extracted from continuous data strings of reflected wave data at an identical position (identical sample point) by using a filter matrix. The signal processing circuitry 120 estimates the bloodstream information by performing calculation such as autocorrelation calculation using the output data, and outputs the estimated bloodstream information as Doppler data.

The image processing circuitry 130 generates ultrasonic image data from the data generated by the signal processing circuitry 120. The image processing circuitry 130 generates, from the two-dimensional B mode data generated by the signal processing circuitry 120, two-dimensional B mode image data in which the intensity of reflected wave is represented as the luminance. The image processing circuitry 130 also generates, from the two-dimensional Doppler data generated by the signal processing circuitry 120, two-dimensional Doppler image data in which the bloodstream information is visualized. The two-dimensional Doppler image data is speed image data, dispersion image data, power image data, or their combination. The image processing circuitry 130 generates the color Doppler image data in which the bloodstream information is displayed in color, and Doppler image data in which one piece of bloodstream information is displayed in gray scale. The image processing circuitry 130 is an exemplary image generation unit.

Typically, the image processing circuitry 130 converts (scanning conversion) a scanning-line signal string of ultrasonic scanning into a scanning-line signal string in a video format for, for example, a television, so as to generate ultrasonic image data. Specifically, the image processing circuitry 130 generates ultrasonic image data by performing coordinate conversion depending on the configuration of ultrasonic scanning by the ultrasonic probe 101. The image processing circuitry 130 also performs various kinds of image processing other than the scanning conversion, such as image processing (smoothing processing) of regenerating an averaged luminance value image by using a plurality of image frames after the scanning conversion, and image processing (edge enhancement processing) using a differential filter in an image. The image processing circuitry 130 also synthesizes the ultrasonic image data with, for example, text information on various kinds of parameters, a scale, and a body mark.

In other words, the B mode data and the Doppler data are ultrasonic image data before the scanning conversion, and the data generated by the image processing circuitry 130 is ultrasonic image data after the scanning conversion. The B mode data and the Doppler data are also called raw data. The image processing circuitry 130 generates two-dimensional ultrasonic image data from two-dimensional ultrasonic image data before the scanning conversion.

In addition, the image processing circuitry 130 generates three-dimensional B mode image data by performing coordinate conversion of the three-dimensional B mode data generated by the signal processing circuitry 120. The image processing circuitry 130 also generates three-dimensional Doppler image data by performing coordinate conversion of the three-dimensional Doppler data generated by the signal processing circuitry 120.

In addition, the image processing circuitry 130 performs rendering processing on volume data to generate various kinds of two-dimensional image data for displaying the volume data on the display 103. The rendering processing performed by the image processing circuitry 130 includes processing of generating MPR image data from the volume data by performing multi planer reconstruction (MPR). Alternatively, the rendering processing performed by the image processing circuitry 130 includes volume rendering (VR) processing of generating two-dimensional image data on which three-dimensional information is reflected.

The image memory 140 stores therein ultrasonic image data generated by the image processing circuitry 13C. The image memory 140 may also store therein data generated by the signal processing circuitry 120. The B mode data and the Doppler data stored in the image memory 140 can be read out by an operator, for example, after diagnosis, and, when read out, is generated as ultrasonic image data through the image processing circuitry 130. The image memory 140 can also store therein reflected wave data output from the transmission and reception circuitry 110.

The storage circuitry 150 stores therein a control program for performing ultrasonic wave transmission and reception, image processing, and display processing, diagnosis information (for example, a patient ID, and findings by a doctor), and various kinds of data such as diagnosis protocols and various body marks. The storage circuitry 150 is used for, for example, storage of image data stored in the image memory 140 as necessary. Data stored in the storage circuitry 150 can be forwarded to an external device through an interface (not illustrated). The storage circuitry 150 can also store therein data forwarded from an external device through the interface (not illustrated).

The processing circuitry 160 controls the entire processing at the ultrasonic diagnostic device 1. Specifically, the processing circuitry 160 controls processing at the transmission and reception circuitry 110, the signal processing circuitry 120, and the image processing circuitry 130 on the basis of various setting requests input by an operator through the input device 102, and various control programs and various kinds of data read from the storage circuitry 150. In other words, the processing circuitry 160 directly or indirectly controls each processes executed by the ultrasonic diagnostic device 1. For example, the processing circuitry 160 controls generating the color Doppler image.

The processing circuitry 160 performs such control that ultrasonic image data stored in the image memory 14C and the storage circuitry 150 is displayed as a display ultrasonic image on the display 103. For example, the processing circuitry 160 displays color Doppler image data generated by the image processing circuitry 130 as a display color Doppler image on the display 103. For example, the processing circuitry 160 displays B mode data generated by the image processing circuitry 130 as a display B mode image on the display 103.

The processing circuitry 160 executes a setting function 161 and a calculation function 162. Processing functions executed by the setting function 161 and the calculation function 162 as components of the processing circuitry 160 are recorded, for example, as computer programs executable by a computer in the storage circuitry 150. The processing circuitry 160 is a processor configured to achieve a function corresponding to each computer program by reading the computer program from the storage circuitry 150 and executing the computer program. In other words, the setting function 161 is achieved by the processing circuitry 160 reading the computer program corresponding to the setting function 161 from the storage circuitry 150 and executing the computer program. The calculation function 162 is achieved by the processing circuitry 160 reading the computer program corresponding to the calculation function 162 from the storage circuitry 150 and executing the computer program. Accordingly, having read the computer programs, the processing circuitry 160 has functions indicated in the processing circuitry 160 illustrated in FIG. 1. The processing functions executed by the setting function 161 and the calculation function 162 will be described later.

In the description of the present embodiment, the above-described processing functions are achieved by the single processing circuitry 160, but the processing circuitry may be configured as a combination of a plurality of independent processors, each processor being configured to achieve a function by executing a computer program.

“Processor” used in the above description represents, for example, a central processing unit (CPU), a graphics processing unit (GPU), or a circuit such as an application specific integrated circuit (ASIC), a programmable logic device (for example, a simple programmable logic device (SPLD)), a complex programmable logic device (CPLD), or a field programmable gate array (FPGA)). A processor achieves a function by reading and executing a computer program stored in the storage circuitry 150. The computer program may be directly incorporated in a circuit of the processor instead of being stored in the storage circuitry 150. In this case, the processor achieves the function by reading and executing the computer program incorporated in the circuit. In the present embodiment, each processor does not necessarily need to be configured as a single circuit, but may be configured as a combination of a plurality of independent circuits to achieve their functions. In addition, a plurality of components in each drawing may be integrated as one processor to achieve their functions.

The above describes the basic configuration of the ultrasonic diagnostic device 1 according to the first embodiment. With this configuration, the ultrasonic diagnostic device 1 according to the first embodiment can achieve an improved display frame rate of a color Doppler image through processing described below.

For example, the processing circuitry 160 controls the ultrasonic probe 101 through the transmission and reception circuitry 110 to perform ultrasonic scanning by the CFM method. Then, the processing circuitry 160 displays a color Doppler image as a bloodstream image and a B mode image as a tissue image on the display 103 on the basis of ultrasonic image data collected through the ultrasonic scanning by the CFM method.

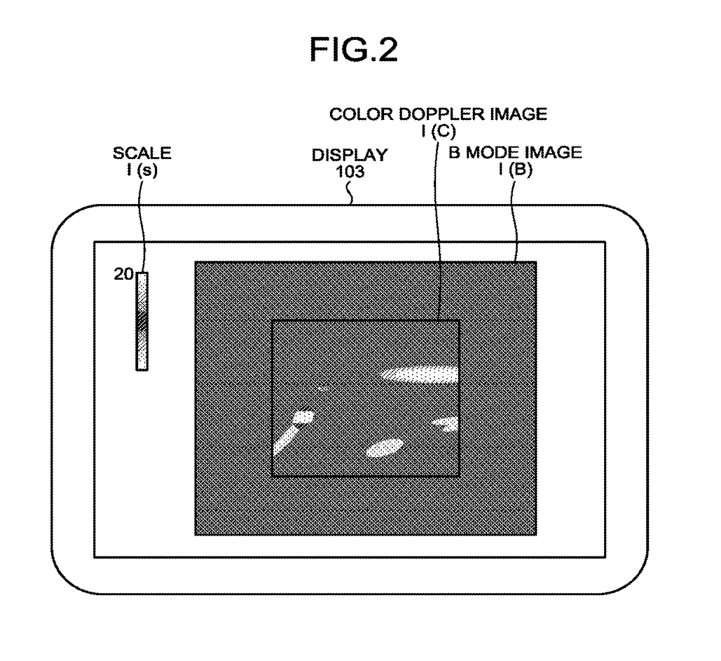

FIG. 2 is a diagram illustrating an exemplary display image displayed by processing at the processing circuitry 160 according to the first embodiment. FIG. 2 exemplarily illustrates a display image displayed by the CFM method.

As illustrated in FIG. 2, for example, the processing circuitry 160 displays color Doppler image I(C) superimposed on B mode image I(B) as a background image on the display 103. In color Doppler image I(C), a color in accordance with, for example, the flow speed value and the direction of bloodstream in a region of interest is allocated at each pixel position, and B mode image I(B) as the background image is displayed at a pixel position having no bloodstream information. The range (hereinafter also referred to as a “flow speed range”) of the flow speed value represented as color Doppler image I(C) is defined with respect to, for example, a maximum flow speed value (upper limit of the flow speed value represented as color Doppler image I(C)) identifiable by color Doppler mode scanning. A correspondence relation between the color drawn in color Doppler image I(C) and the flow speed value is indicated by scale I(s). FIG. 2 exemplarily illustrates scale I(s) for a maximum detected flow speed of 20 [cm/sec]. Although FIG. 2 exemplarily illustrates B mode image I(B) in a single color for sake of simplicity of description, a tissue image is drawn instead in reality.

In order to display a display image by the CFM method, the transmission and reception circuitry 110 executes color Doppler mode scanning to collect color Doppler image data, and B mode scanning to collect B mode data. Specifically, in order to display a display image of one frame exemplarily illustrated in FIG. 2, the transmission and reception circuitry 110 executes color Doppler mode scanning of a region of interest and B mode scanning of a region larger than the region of interest. The color Doppler mode scanning is an exemplary first scanning, and the B mode scanning is an exemplary second scanning. The transmission and reception circuitry 110 is an exemplary transmission and reception unit.

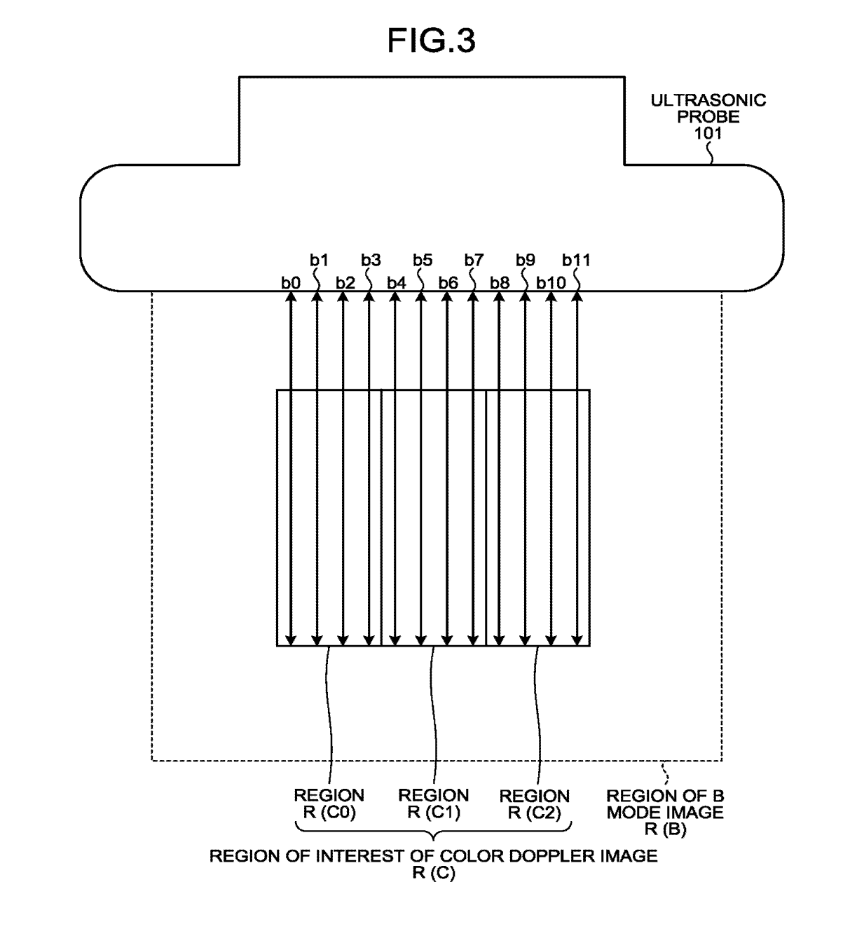

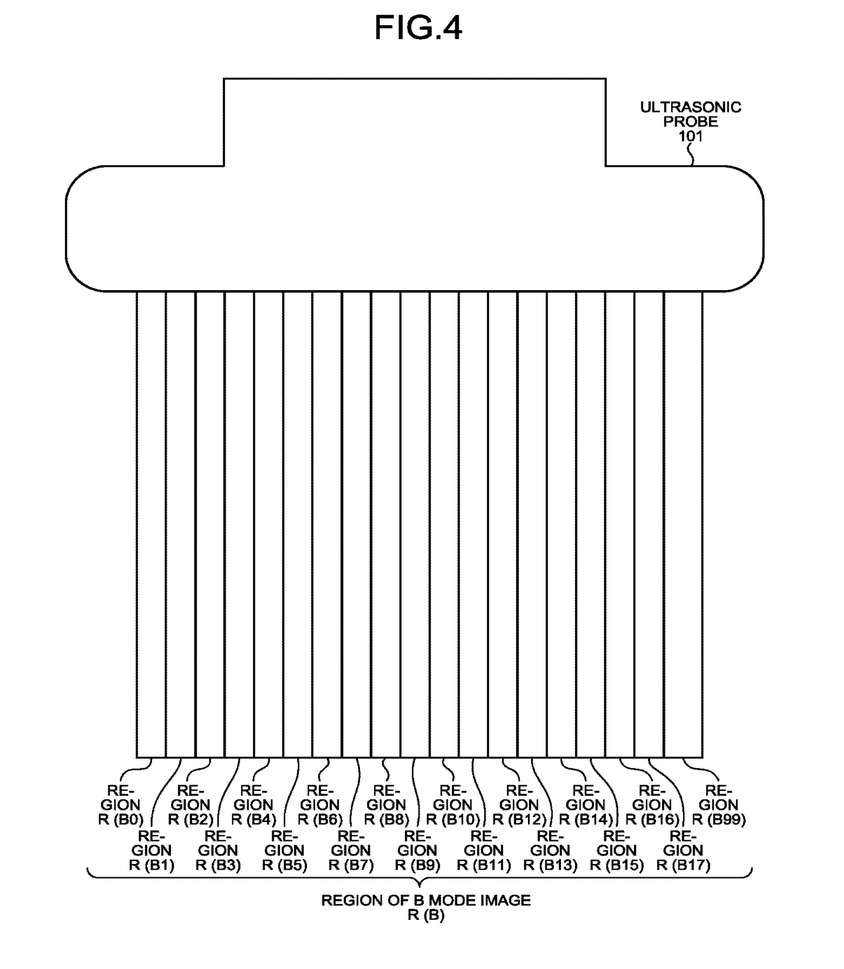

FIGS. 3 and 4 are diagrams for describing a region scanned by t transmission and reception circuitry 110 according to the first embodiment. FIG. 3 exemplarily illustrates region of interest R(C) in which color Doppler image data is collected. FIG. 4 exemplarily illustrates region R(B) in which B mode image data is collected. Region of interest R(C) corresponds to the region of color Doppler image I(C) illustrated in FIG. 2, and region R(B) corresponds to the region of B mode image I(B) illustrated in FIG. 2.

As illustrated in FIG. 3, the transmission and reception circuitry 110 executes the color Doppler mode scanning of region of interest R(C) to collect, for example, color Doppler image data of one frame. This region of interest R(C) includes, for example, twelve beams (scanning lines) transmitted and received at beam positions b0 to b11. Specifically, the transmission and reception circuitry 110 divides region of interest R(into three regions of region R(C0), region R(C1), and region R(C2) for scanning. Regions R(C0) to R(C2) each include, for example, four beams.

In the CFM method, a data string of reflected wave data at an identical position is used to generate bloodstream information of one frame. For this purpose, the transmission and reception circuitry 110 collects a data string at each position (sample point) in region of interest R(C) by repeatedly executing the color Doppler mode scanning of region of interest R(C). For example, the transmission and reception circuitry 110 collects color Doppler image data of one frame by executing the color Doppler mode scanning of region of interest R(C) six times in a predetermined repetition period. In the example illustrated in FIG. 3, the transmission and reception circuitry 110 executes the color Doppler mode scanning six times for each of divided regions R(C0) to R(C2). In other words, the repetition period corresponds to a period in which the first scanning (the color Doppler mode scanning) is repeated.

As illustrated in FIG. 4, the transmission and reception circuitry 110 executes the B mode scanning of region R(B) to collect B mode data of one frame. For example, the transmission and reception circuitry 110 scans 19 divided regions of region R(B). Specifically, the transmission and reception circuitry 110 divides region R(B) into region R(B0), region R(B1), region R(B2), region R(B3), region R(B4), region R(B5), region R(B6), region R(B7), region R(B8), region R(B9), region R(B10), region R(B11), region R(B12), region R(B13), region R(B14), region R(B15), region R(B16), region R(B17), and region R(B99), and executes the B mode scanning of region R(B).

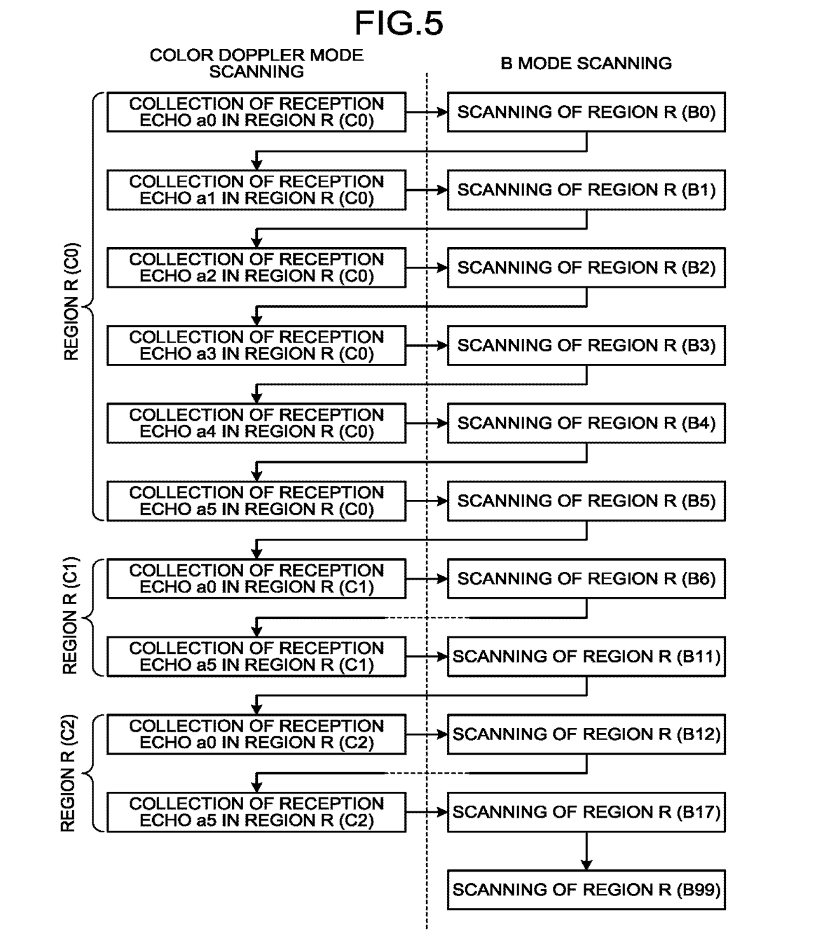

In this manner, the transmission and reception circuitry 110 executes the color Doppler mode scanning of regions R(C0) to R(C2) and the B mode scanning of regions R(B0) to R(B99) to display a display image of one frame exemplarily illustrated in FIG. 2. An order of the scanning of regions R(C0) to R(C2) and regions R(B0) to R(B99) by the transmission and reception circuitry 110 will be described later with reference to FIG. 5.

FIGS. 3 and 4 are merely examples. For example, FIGS. 3 and 4 exemplarily illustrate a case in which region of interest R(C) is smaller than region R(B), but embodiments are not limited thereto. For example, the dimensions (widths in the azimuth direction, and depths in the depth direction) of region of interest R(C) and region R(B) may be optionally set. However, the dimension of region of interest R(C) is preferably the same as that of region R(B) as a background image or smaller than that of region R(B).

FIG. 5 is a diagram for describing the order of scanning by the transmission and reception circuitry 110 according to the first embodiment. FIG. 5 illustrates, with arrows, the order of scanning the regions illustrated in FIGS. 3 and 4. The color Doppler mode scanning is illustrated on the left side in FIG. 5, and the B mode scanning is illustrated on the right side in FIG. 5. FIG. 5 exemplarily illustrates region of interest R(C) with a packet size of “6”. In the following, pieces of reflected wave data for six times of execution included in a packet are referred to as reception echoes a0 to a5.

As illustrated in FIG. 5, for example, the transmission and reception circuitry 110 collects reception echo a0 of region R(C0), and executes scanning of region R(B0) after the collection of reception echo a0. Next, the transmission and reception circuitry 110 collects reception echo a1 of region R(C0), and executes scanning of region R(B1) after the collection of reception echo a1. Subsequently, the transmission and reception circuitry 110 collects reception echo a2 of region R(C0), and executes scanning of region R(B2) after the collection of reception echo a2. Thereafter, the transmission and reception circuitry 110 collects reception echo acs of region R(C0), and executes scanning of region R(B3) after the collection of reception echo a3. Thereafter, the transmission and reception circuitry 110 collects reception echo a1 of region R(C0), and executes scanning of region R(B4) after the collection of reception echo a4. Thereafter, the transmission and reception circuitry 110 collects reception echo a5 of region R(C0), and executes scanning of region R(B5) after the collection of reception echo a5.

In this manner, the transmission and reception circuitry 110 collects reception echoes a0 to a5 of region R(C0), and executes scanning of each of regions R(B0) to R(B5) after the collection of the corresponding one of reception echoes a0 to a5. The transmission and reception circuitry 110 scans region R(C1) and region R(C2) in a similar manner to that of region R(C0).

Specifically, the transmission and reception circuitry 110 collects reception echoes a0 to a5 of region R(C1), and executes scanning of each of regions R(B6) to R(B11) after the collection of the corresponding one of reception echoes a0 to a5. The transmission and reception circuitry 110, collects reception echoes a0 to a5 of region R(C2), and executes scanning of each of regions R(B12) to R(B17) after the collection of the corresponding one of reception echoes a0 to a5. Thereafter, the transmission and reception circuitry 110 executes scanning of region R(B99).

In this manner, the transmission and reception circuitry 110 collects reception echoes of regions corresponding to color Doppler image I(C), and executes scanning of each of regions corresponding to B mode image I(B) after the collection of the corresponding reception echo.

FIG. 5 is merely an example. For example, FIG. 5 illustrates the case in which the color Doppler mode scanning is performed in an order of region R(C0), region R(C1), and region R(C2), but this order is optionally changeable. For example, FIG. 5 illustrates the case in which the B mode scanning is performed in an order of regions R(B0) to (B17), but this order is optionally changeable.

To allow the transmission and reception circuitry 110 to execute scanning of each region in the order of scanning illustrated in FIG. 5, the processing circuitry 160 according to the first embodiment determines a scanning condition defining a condition of the scanning executed by the transmission and reception circuitry 110. Specifically, the processing circuitry 160 determines the scanning condition, and causes the transmission and reception circuitry 110 to execute scanning by the CFM method on the basis of the determined scanning condition. The following describes processing executed by the processing circuitry 160 to determine the scanning condition.

The setting function 161 sets the flow speed range. For example, the setting function 161 sets the maximum detected flow speed of the flow speed range. The setting function 161 is an exemplary setting unit.

For example, at start of scanning by the CFM method, the setting function 161 receives an input to specify the maximum detected flow speed of the flow speed range from an operator. Specifically, the operator performs inputting to specify the maximum detected flow speed by operating a knob or a keyboard on an operation panel of the ultrasonic diagnostic device 1. Then, the setting function 161 sets the flow speed range by using the maximum detected flow speed input by the operator. When the maximum detected flow speed in accordance with the scanning condition is preset, the setting function 161 may set the preset maximum detected flow speed without receiving inputting from the operator.

For example, during scanning by the CFM method, the setting function 161 receives, from the operator, an input to change the maximum detected flow speed of the flow speed range. Then, the setting function 161 changes (resets) the flow steed range with the maximum detected flow speed changed by the operator.

The calculation function 162 calculates a wait time of the color Doppler mode scanning on the basis of a repetition period of the color Doppler mode scanning and a required time of the color Doppler mode scanning. Then, the calculation function 162 causes the transmission and reception circuitry 110 to execute the B mode scanning in the calculated wait time. In other words, the transmission and reception circuitry 110 executes the B mode scanning during the wait time calculated by the calculation function 162. The calculation function 162 is an exemplary calculation unit.

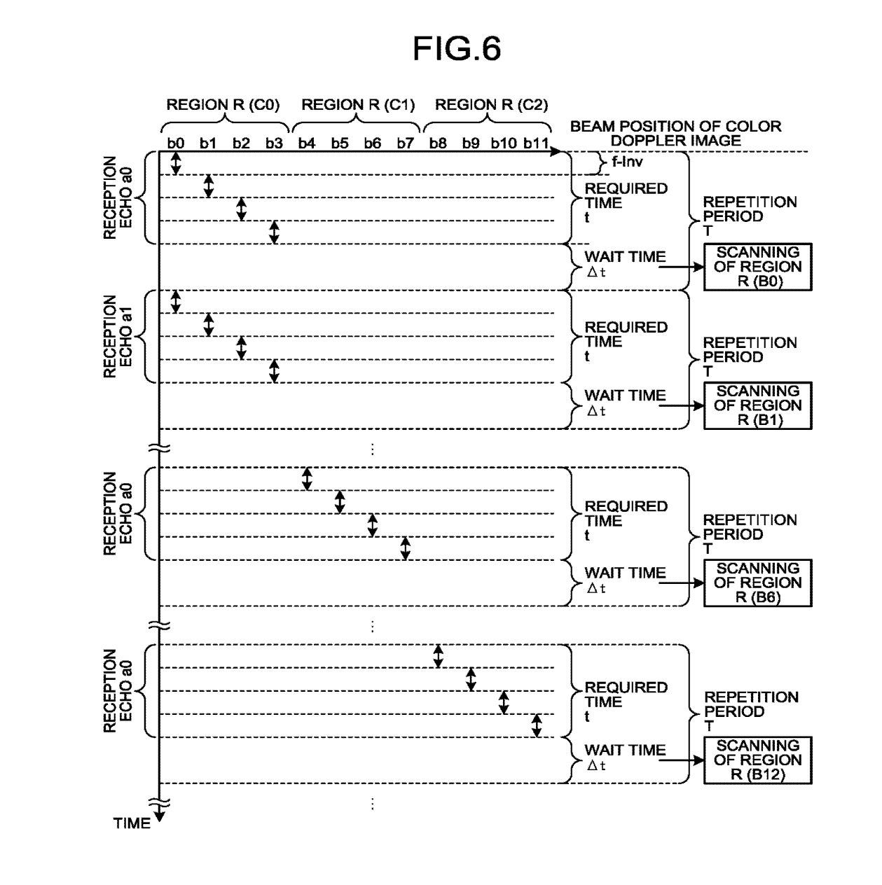

FIG. 6 is a diagram for describing processing of calculating the wait time by the calculation function 162 according the first embodiment. In FIG. 6, the vertical axis represents time, and the horizontal axis represents beam position of the color Doppler mode scanning. For sake of simplicity of description, FIG. 6 exemplarily illustrates a case in which parallel simultaneous reception is not performed. FIG. 6 exemplarily illustrates a case in which the number of beams arranged in region of interest R(C) of the color Doppler mode scanning is 12 (beam positions b0 to b11).

An acoustic pulse repetition frequency (PRF) corresponds to a reciprocal of duration (time) from transmission of a beam until transmission of the next beam. Thus, for example, in reception echo a0 of region R(C0), the reciprocal “f-Inv” of the acoustic PRF corresponds to a time from execution of transmission and reception at beam position b0 until execution of transmission and reception at beam position b1, and thus corresponds to a transmission and reception time taken for transmission and reception of each beam. The acoustic PRE is determined on the basis of, for example, at least one of the position (depth) of a lower end of region of interest R(C), the flow speed range, and the reception frequency of ultrasonic wave.

As illustrated in FIG. 6, the calculation function 162 calculates a repetition period T of the color Doppler mode scanning. The repetition period T corresponds to a duration (time) in which transmission and reception is repeatedly executed at a beam position. Specifically, the repetition period T corresponds to, for example, a time from transmission and reception of reception echo a0 at beam position b0 until transmission and reception of reception echo a1 at beam position b0. The repetition period T is shorter at a higher maximum detected flow speed but longer at a lower maximum detected flow speed. Thus, the calculation function 162 calculates the repetition period T on the basis of the maximum detected flow speed of the flow speed range set by the setting function 161. The repetition period T has an identical value between reception echoes a0 to a5 included in a packet of region R(C).

Next, the calculation function 162 calculates the number of alternation stages of alternate scanning on the basis of the repetition period T and the acoustic PRF. In the alternate scanning, when a data string at an identical position is collected by the CFM method, a group of a plurality of beam positions are set, and transmission and reception is performed sequentially at the beam positions included in the group, instead of performing transmission and reception continuously at an identical beam position. In the example illustrated in FIG. 6, four beams positions b0 to b3 are set as a group, and transmission and reception is executed sequentially at beam positions b0, b1, b2, and b3 corresponding to reception echo a0, and then transmission and reception is executed sequentially at beam positions b0, b1, b2, and b3 corresponding to reception echo a1. In the alternate scanning, the number of beam positions included in each reception echo is called the number of alternation stages. Thus, the number of alternation stages corresponds to the number of beams included in each of regions R(C0) to R(C2). FIG. 6 exemplarily illustrates a case in which the calculation function 162 calculates the number of alternation stages to be “4”.

Subsequently, the calculation function 162 calculates the number of divisions of region of interest R(C) of the color Doppler mode scanning. For example, the calculation function 162 calculates the number of divisions of region of interest R(C) by dividing the number of beams in region of interest R(C) by the number of alternation stages. When a remainder exists, the calculation function 162 calculates the number of divisions of region of interest R(C) by rounding up the solution of the division to an integer. In the example illustrated in FIG. 6, the calculation function 162 calculates the number of divisions of region of interest R(C) to be “3” by dividing the number of beams in region of interest R(C), which is “12”, by the number of alternation stages, which is “4”.

Then, the calculation function 162 calculates a required time t taken for collection of reception echo a0 of region R(C0). The number of beams included in reception echo a0 of region R(C0) is “4”. A time taken for transmission and reception of each beam corresponds to the reciprocal “f-Inv” of the acoustic FRF, and thus the calculation function 162 calculates the required time t by multiplying the reciprocal “f-Inv” of the acoustic PRF by the number of beams “4”. The required time t has an identical value when the number of collected beams is the same for reception echoes a0 to a5. In other words, the required time t corresponds to a minimum necessary time for scanning along a plurality of scanning lines (beams).

Then, the calculation function 162 calculates a wait time Δt on the basis of the repetition period T and the required time t. The wait time Δt corresponds to a time from completion of transmission and reception of the last beam in a packet until the start of transmission and reception of the first beam in the next packet. Thus, the wait time Δt corresponds to, for example, a time from transmission and reception of reception echo a0 at beam position b3 until transmission and reception of reception echo a1 at beam position b0. Accordingly, the calculation function 162 calculates the wait time Δt by subtracting the required time t from the repetition period T. In other words, the wait time Δt corresponds to a time from the end of the previous first scanning (the color Doppler mode scanning) until the start of the next first scanning. The repetition period T includes the required time t and the wait time Δt. The wait time Δt is obtained as a difference between the repetition period T and the required time t.

In this manner, the calculation function 162 calculates the wait time Δt. Then, the calculation function 162 calculates the number of beams that can be transmitted and received in the calculated wait time Δt in the B mode scanning. For example, the calculation function 162 calculates the number of beams that can be transmitted and received during the wait time Δt in the B mode scanning on the basis of the calculated wait time Δt and a time taken for transmission and reception to and from a lower end of region R(B) of the B mode scanning.

Then, the calculation function 162 determines the scanning condition on the basis of the various calculated parameters. Specifically, the calculation function 162 sequentially allocates the B mode scanning with the calculated number of beams to the calculated wait time Δt of each reception echo so as to determine the corresponding one of B mode regions R(B0) to R(B17) to be scanned after the reception echo.

In this manner, the calculation function 162 determines the order of scanning the regions of the color Doppler mode scanning and the B mode scanning. The calculation function 162 sets the scanning condition so that the regions are scanned in the determined order of scanning. Then, the calculation function 162 causes the transmission and reception circuitry 110 to execute ultrasonic scanning on the basis of the set scanning condition. Accordingly, for example, the transmission and reception circuitry 110 executes scanning of the regions of the color Doppler mode scanning and the B mode scanning in the order of scanning illustrated in FIG. 5.

FIG. 6 is merely an example. For example, FIG. 6 illustrates the case in which the calculation of the number of divisions of region of interest R(C) yields no remainder, but embodiments are not limited thereto. Processing in a case in which a remainder exists will be described later as a first modification of the first embodiment.

For example, in the above description, the calculation function 162 calculates the wait time each time the scanning condition is set, but embodiments are not limited thereto. For example, the calculation function 162 does not necessarily need to calculate the wait time. For example, the calculation function 162 may obtain a wait time calculated in advance. For example, the calculation function 162 may obtain a wait time from a table storing therein a wait time calculated in advance depending on a particular scanning condition. As a specific example, when the scanning condition (including the repetition period and the required time described above) is set in advance for each imaged site, the calculation function 162 calculates in advance a wait time depending on the scanning condition for the imaged site, and stores the calculated wait time in association with the imaged site in a table. Then, when an imaged site to be scanned is specified, the calculation function 162 reads the wait time corresponding to the specified imaged site from the table. Then, for example, the calculation function 162 determines the order of scanning of the regions of the color Doppler mode scanning and the B mode scanning by using the read wait time. In other words, the calculation function 162 serves as an acquisition unit configured to obtain a wait time. In other words, the processing circuitry 160 controls obtaining the wait time.

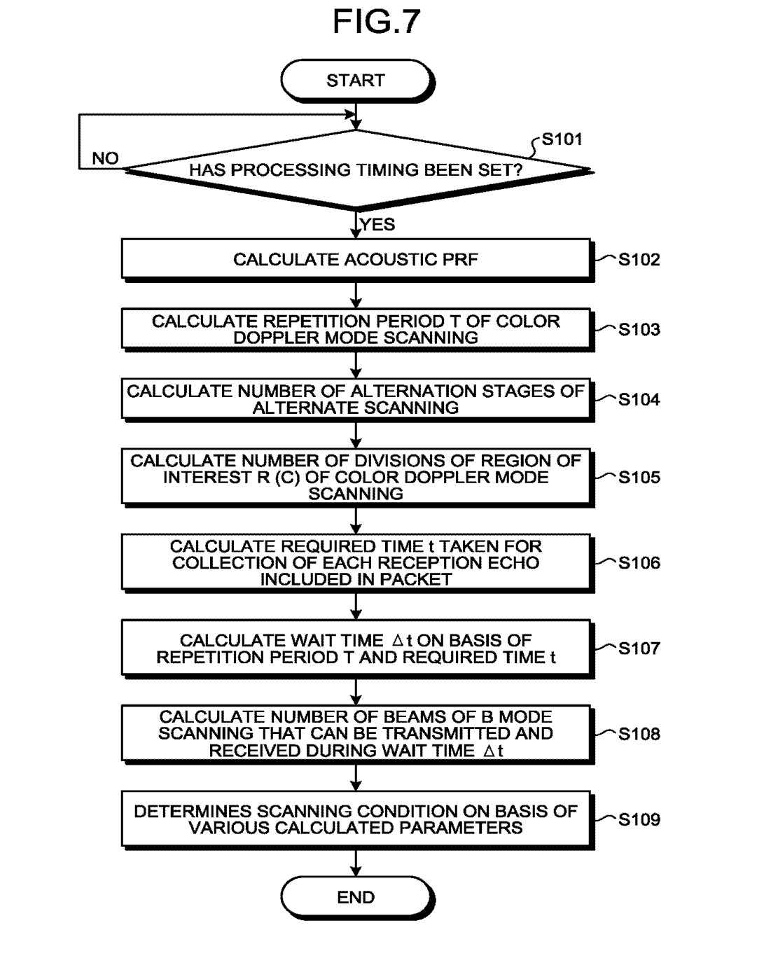

FIG. 7 is a flowchart the procedure of processing at the processing circuitry 160 according to the first embodiment. The procedure of processing illustrated in FIG. 7 is started, for example, when the setting function 161 sets the flow speed range.

At step S101, it is determined whether the setting function 161 has set the flow speed range. If the setting function 161 has set the flow speed range (Yes at step S101), the processing circuitry 160 starts processing at step S102 and later. If the determination at step S101 is negative, the processing at step S102 and later does not start, and each processing function of the processing circuitry 160 is in a waiting state.

If the determination step S101 is positive, the calculation function 162 calculates the acoustic PRF at step S102. For example, the calculation function 162 calculates the acoustic PRF on the basis of the position (depth) of the lower end of region of interest R(C) of the color Doppler mode scanning and the reception frequency of ultrasonic wave.

At step S103, the calculation function 162 calculates the repetition period T of the color Doppler mode scanning. For example, the calculation function 162 calculates the repetition period T on the basis of the maximum detected flow speed of the flow speed range set by the setting function 161.

At step S104, the calculation function 162 calculates the number of alternation stages of the alternate scanning. The number of alternation stages corresponds to the number of beams included in each of regions R(C0) to R(C2). For example, the calculation function 162 calculates the number of alternation stages of the alternate scanning on the basis of the repetition period T and the acoustic PRF.

At step S105, the calculation function 162 calculates the number of divisions of region of interest R(C) of the color Doppler mode scanning. For example, the calculation function 162 calculates the number of divisions of region of interest R(C) by dividing the number of beams in region of interest R(C) by the number of alternation stages.

At step S106, the calculation function 162 calculates the required time t taken for collection of each reception echo included in a packet. For example, the calculation function 162 calculates the required time t by multiplying the reciprocal “f-Inv” of the acoustic PRE by the number of alternation stages (number of beams).

At step S107, the calculation function 162 calculates the wait time Δt on the basis of the repetition period T and the required time t. For example, the calculation function 162 calculates the wait time Δt by subtracting the required time t from the repetition period T.

At step S108, the calculation function 162 calculates the number of beams of the B mode scanning that can be transmitted and received during the wait time Δt. For example, the calculation function 162 calculates the number of beams of the B mode scanning that can be transmitted and received during the wait time Δt on the basis of the calculated wait time Δt and the time taken for transmission and reception to and from the lower end of region R(B) of the B mode scanning.

At step S109, the calculation function 162 determines the scanning condition on the basis of the various calculated parameters. For example, the calculation function 162 sequentially allocates the B mode scanning with the calculated number of beams to the calculated wait time Δt of each reception echo so as to determine the corresponding one of B mode regions R(B0) to R(B17) scanned after the reception echo. Then, the calculation function 162 sets the scanning condition so that the regions are scanned in the determined order of scanning. As a result, the transmission and reception circuitry 110 executes scanning of the regions of the color Doppler mode scanning and the B mode scanning in the order of scanning set by the scanning condition.

As described above, in the ultrasonic diagnostic device 1 according to the first embodiment, the transmission and reception circuitry 110 repeatedly executes the color Doppler mode scanning. The image processing circuitry 130 generates color Doppler image I(C) on the basis of the reflected wave data (reception echo) collected through the color Doppler mode scanning. The calculation function 162 calculates the wait time Δt of the color Doppler mode scanning on the basis of the repetition period T of the color Doppler mode scanning and the required time t of the color Doppler mode scanning. The transmission and reception circuitry 110 executes the B mode scanning during the wait time Δt calculated by the calculation function 162. Accordingly, the ultrasonic diagnostic device 1 according to the first embodiment can display color Doppler image I(C) at a high frame rate.

For example, in the CFM method, when bloodstream at a low flow speed is to be observed, an operator sets a low maximum detected flow speed of the flow speed range. In this case, bloodstream information at a low flow speed has been imaged at a reduced frame rate in the conventional CFM method because of increase in the repetition period T. However, the ultrasonic diagnostic device 1 according to the first embodiment executes the B mode scanning during the wait time Δt generated due to the increase in the repetition period T. Accordingly, the ultrasonic diagnostic device 1 can prevent reduction in the frame rate, for example, when displaying a color Doppler image at a low flow speed.

The number of alternation stages of the alternate scanning needs to be reduced in some cases because of, for example, limit of a memory capacity for temporarily storing therein packets. In such a case, in order to perform scanning of the same region of interest R(C), the number of divisions of the region needs to be increased, which results in reduction in the frame rate in the conventional CFM method. However, the ultrasonic diagnostic device 1 according to the first embodiment executes the B mode scanning during the wait time Δt generated by reduction in the required time t due to reduction in the number of alternation stages. With this configuration, the ultrasonic diagnostic device 1 can achieve an improved frame rate, for example, even when the memory capacity is limited. Accordingly, the ultrasonic diagnostic device 1 can achieve improved visibility of an ultrasonic image.

For example, the ultrasonic diagnostic device 1 according to the first embodiment executes the color Doppler mode scanning and the B mode scanning at an identical frame rate. This allows the ultrasonic diagnostic device 1 to set the scanning condition of the color Doppler mode scanning and the B mode scanning as appropriate, thereby achieving enhanced freedom of the scanning condition.

### First Modification of First Embodiment

Although the first embodiment describes the case in which the calculation of the number of divisions of region of interest yields no remainder, a remainder is obtained in some cases. The following describes processing executed when a remainder is obtained in the calculation of the number of divisions.

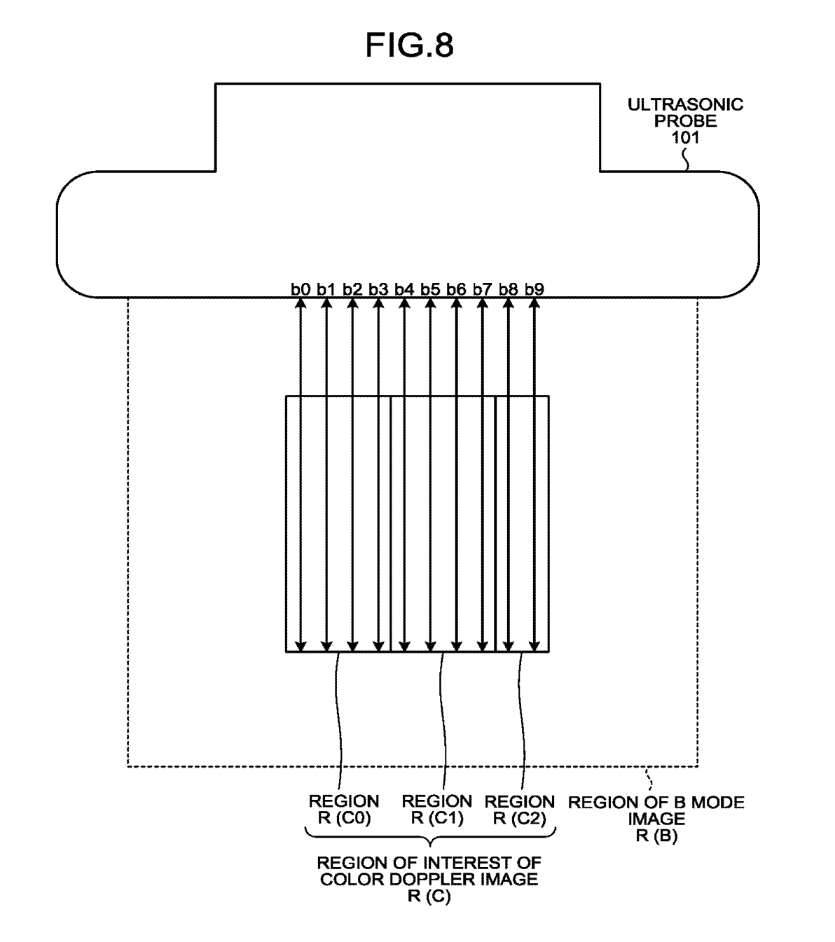

FIG. 8 is a diagram for describing a region scanned by the transmission and reception circuitry 110 according to the first modification of the first embodiment. FIG. 8 exemplarily illustrates region of interest R(C) in which color Doppler image data is collected. The region of interest R(C) illustrated in FIG. 8 includes 10 beams (scanning line) transmitted and received at beam positions b0 to b9.

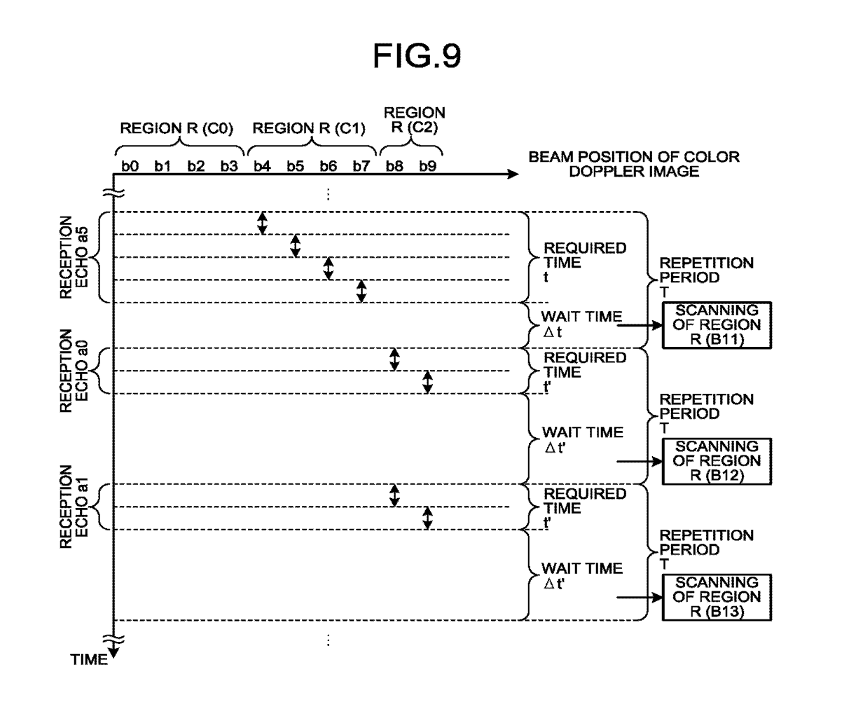

FIG. 9 is a diagram for describing processing of calculating the wait time at the calculation function 162 according to the first modification of the first embodiment. In FIG. 9, the vertical axis corresponds to time, and the horizontal axis corresponds to the beam position of the color Doppler mode scanning. For sake of simplicity of description, FIG. 9 exemplarily illustrates a case in which parallel simultaneous reception is not performed. FIG. 9 illustrates the processing of calculating the wait time in region of interest R(C) illustrated in FIG. 8. Processing of calculating the acoustic PRF, the repetition period T, and the number of alternation stages in FIG. 9 is the same as that illustrated in FIG. 6.

As illustrated in FIG. 9, the calculation function 162 obtains “2.5” by dividing the number of beams in region of interest R(C), which is “10” by the number of alternation stages, which is “4”. When a remainder exists (the solution of the division has any digit after the decimal point), the calculation function 162 calculates the number of divisions of region of interest R(C) to be “3” by rounding up the solution to an integer.

When a remainder exists, the number of beams included in each of region R(C0) and region R(C1) is “4”, whereas the number of beams included in region R(C2) is “2”. In this case, the required time t and the wait time Δt can be calculated for region R(C0) and region R(C1) in a similar manner to that of the case illustrated in FIG. 6.

However, a required time t′ calculated in region R(C2) is shorter than the required time t. Specifically, the calculation function 162 calculates the required time t′ by multiplying the reciprocal “f-Inv” of the acoustic PRF by the number of beams “2”.

Thus, for region R(C2), the required time t′ is shorter than the required time t, but the repetition period T is the same as that for region R(CC) and region R(C1). Accordingly, a wait time Δt′ in region R(C2) is longer than the wait time Δt. Specifically, the calculation function 162 calculates the wait time Δt′ by subtracting the required time t′ from the repetition period T.

Then, the calculation function 162 calculates the number of beams of the B mode scanning that can be transmitted and received in the calculated wait time Δt′. This number of beams is larger than the number of beams that can be transmitted and received during the wait time Δt, which enables scanning of a larger region. In the example illustrated in FIG. 9, region R(B12) and region R(B13) scanned during the wait time Δt′ can be larger than region R(B11) scanned during the wait time Δt.

In this manner, when the calculation of the number of divisions of region of interest R(C) yields a remainder, there exists a region for which the number of alternation stages is smaller than for any other region. In this case, the wait time Δt′ is longer in the region in which the number of alternation stages is smaller, which allows increase in the region of the B mode scanning that can be scanned during the wait time Δt′.

### Second Modification of First Embodiment

Although the first embodiment describes the case in which scanning is not completed within all of the wait Δt during the color Doppler mode scanning, the B mode scanning is completed within all of the wait time Δt in some cases. In such a case, region R(B99) of the B mode scanning does not necessarily exist.

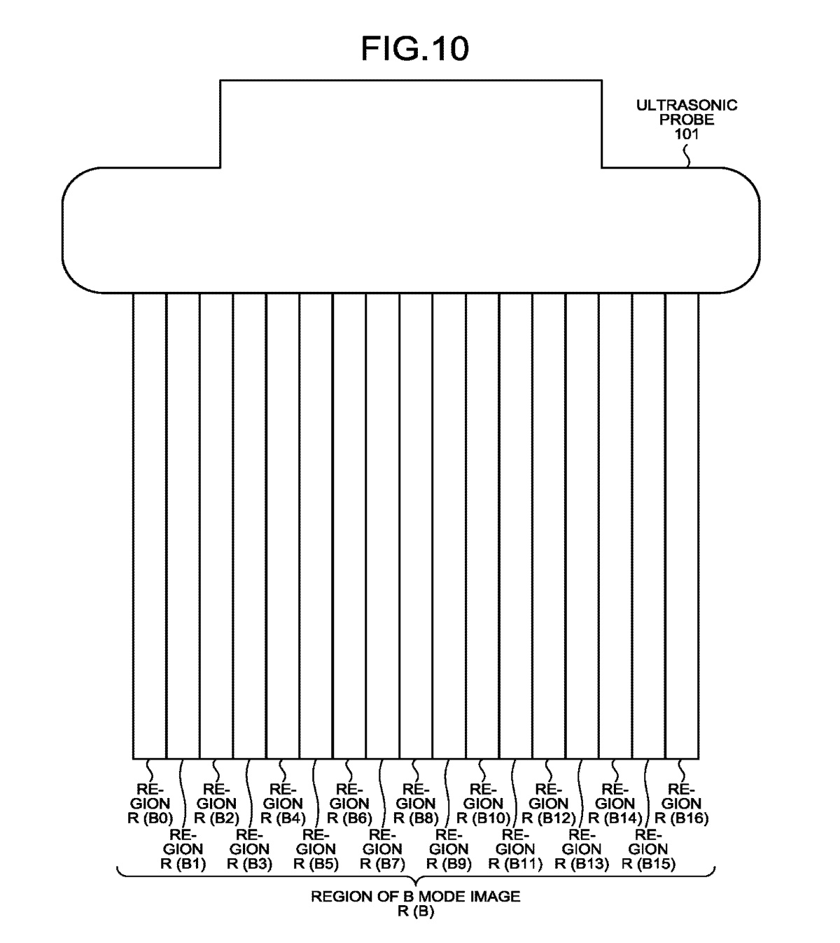

FIG. 10 is a diagram for describing a region scanned by the transmission and reception circuitry 110 according to a second modification of the first embodiment. FIG. 10 exemplarily illustrates region R(B) in which B mode image data is collected.

As illustrated in FIG. 10, the transmission and reception circuitry 110 executes the B mode scanning of region R(B) to collect B mode data of one frame. For example, the transmission and reception circuitry 110 scans 17 divided regions of region R(B). Specifically, the transmission and reception circuitry 110 divides region R(B) into region R(B0), region R(B1), region R(B2), region R(B3), region R(B4), region R(B5), region R(B6), region R(B7), region R(B8), region R(B9), region R(B10), region R(B11), region R(B12), region R(B13), region R(B14), region R(B15), and region R(B16), and executes the B mode scanning of region R(B).

In this manner, the transmission and reception circuitry 110 executes the color Doppler mode scanning of regions R(C0) to R(C2) and the B mode scanning of regions R(B0) to R(B16) to display the display image of one frame exemplarily illustrated in FIG. 2. In other words, when a time taken for the B mode scanning is shorter than the total of all wait times of the color Doppler mode scanning, the calculation function 162 sets a time during which no B mode scanning is executed in any of the wait times.

### Second Embodiment

Although the above-described embodiment describes the case in which the visibility of an ultrasonic image is improved through improvement of the frame rate, the present embodiment is not limited thereto. The visibility of an ultrasonic image may be improved through, for example, improvement of the image quality of the ultrasonic image. The second embodiment thus describes a case in which the visibility of an ultrasonic image is improved through improvement of the image quality of the ultrasonic image.

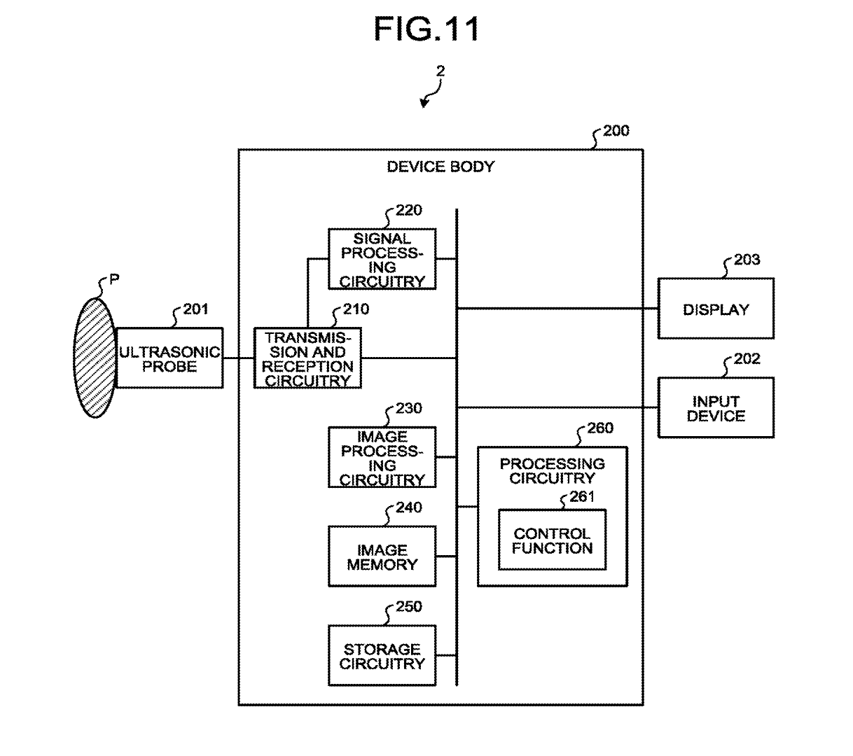

FIG. 11 is a block diagram of an exemplary configuration of an ultrasonic diagnostic device 2 according to the second embodiment. As illustrated in FIG. 11, the ultrasonic diagnostic device 2 according to the second embodiment includes an ultrasonic probe 201, an input device 202, a display 203, and a device body 200. The ultrasonic probe 201, the input device 202, and the display 203 are connected with the device body 200 to perform communication therebetween. The subject P is not included in the configuration of the ultrasonic diagnostic device 2.

The ultrasonic probe 201, the input device 202, and the display 203 are the same as the ultrasonic probe 101, the input device 102, and the display 103 illustrated in FIG. 1, respectively, and thus descriptions thereof are omitted.

The device body 200 is a device configured to generate ultrasonic image data on the basis of a reflected wave signal received by the ultrasonic probe 201. As exemplarily illustrated in FIG. 11, the device body 200 includes a transmission and reception circuitry 210, signal processing circuitry 220, image processing circuitry 230, an image memory 240, storage circuitry 250, and processing circuitry 260. The transmission and reception circuitry 210, the signal processing circuitry 220, the image processing circuitry 230, the image memory 240, the storage circuitry 250, and the processing circuitry 260 are connected with each other to perform communication therebetween.

The transmission and reception circuitry 210, the signal processing circuitry 220, the image processing circuitry 230, the image memory 240, and the storage circuitry 250 are the same as the transmission and reception circuitry 110, the signal processing circuitry 120, the image processing circuitry 130, the image memory 140, and the storage circuitry 150 illustrated in FIG. 1, respectively, and thus descriptions thereof are omitted.

Similarly to the processing circuitry 160 illustrated in FIG. 1, the processing circuitry 260 controls the entire processing at the ultrasonic diagnostic device 2. For example, the processing circuitry 260 controls processing at the transmission and reception circuitry 210, the signal processing circuitry 220, and the image processing circuitry 230 on the basis of various setting requests input by an operator through the input device 202, and various control programs and various kinds of data read from the storage circuitry 250. In other words, the processing circuitry 260 directly or indirectly controls each processes executed by the ultrasonic diagnostic device 2. For example, the processing circuitry 260 controls generating the color Doppler image.

For example, the processing circuitry 260 performs such control that ultrasonic image data stored in the image memory 240 and the storage circuitry 250 is displayed as a display ultrasonic image on the display 203. For example, the processing circuitry 260 displays color Doppler image data generated by the image processing circuitry 230 as a display color Doppler image on the display 203. For example, the processing circuitry 260 displays B mode data generated by the image processing circuitry 230 as a display B mode image on the display 203.

The processing circuitry 260 according to the second embodiment executes a control function 261. A processing function executed by the control function 261 as a component of the processing circuitry 260 is recorded, for example, as a computer program executable by a computer in the storage circuitry 250. The processing circuitry 260 is a processor configured to achieve a function corresponding to each computer program by reading the computer program from the storage circuitry 250 and executing the computer program. “Processor” in the above description represents what is represented by “processor” described in the first embodiment.

The control function 261 according to the second embodiment controls the transmission and reception circuitry 210 so that scanning is executed with ultrasonic wave having energy in accordance with the length of a wait time corresponding to a time from the end of the previous scanning until the start of the next scanning. During the wait time, the control function 261 reduces electrical power supplied to an electronic circuit included in the ultrasonic probe 201 including a transducer element.

For example, the control function 261 sets a wait time to be a time during which no scanning is executed, and executes scanning with ultrasonic wave having energy in accordance with the length of the set wait time. For example, during the wait time, the control function 261 reduces electrical power supplied to the electronic circuit included in the ultrasonic probe 201 that performs scanning.

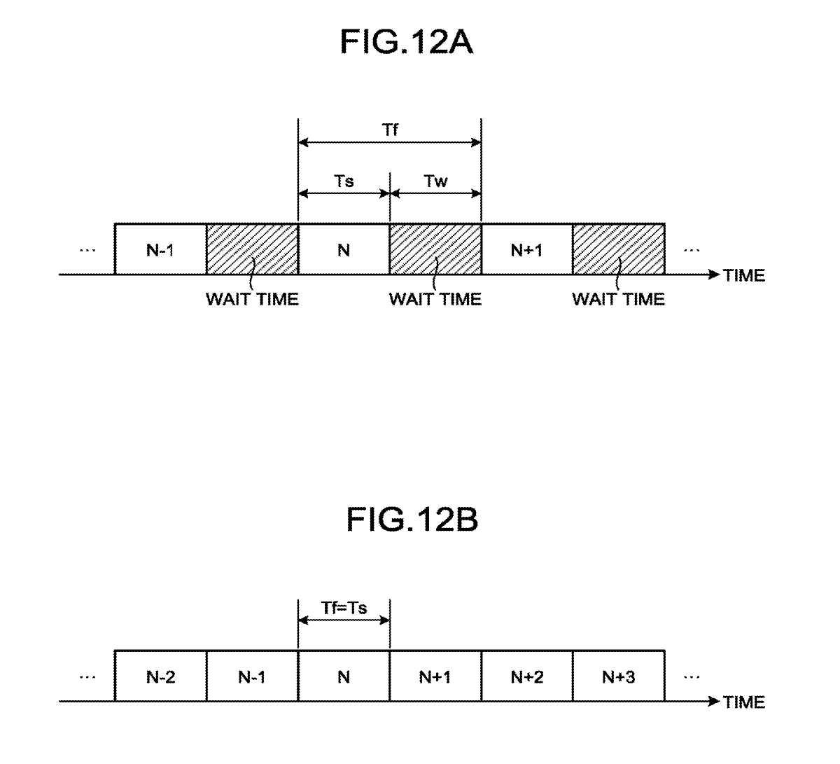

FIGS. 12A and 12B are diagrams illustrating processing at the control function 261 according to the second embodiment. FIG. 12A exemplarily illustrates a timing chart of scanning executed by the processing at the control function 261. FIG. 12B exemplarily illustrates a timing chart when the processing at the control function 261 is not applied. FIGS. 12A and 12B illustrate a case in which the B mode scanning is executed as exemplary scanning.

As illustrated in FIG. 12A, for example, the control function 261 sets, between frames of scanning, each wait time during which no scanning is executed. In the example illustrated in FIG. 12A, the control function 261 sets such a wait time between scanning in the (N−1)-th frame and scanning in the N-th frame. The control function 261 also sets another wait time between scanning in the N-th frame and scanning in the (N+1)-th frame. The control function 261 also sets another wait time between scanning in the (N+1)-th frame and scanning in the (N+2)-th frame (not illustrated).

When a required time for scanning in each frame is “Ts” seconds and a wait time is “Tw” seconds, an inter-frame time interval (corresponding to the reciprocal of the frame rate) is “Tf=Ts+Tw”.

Then, the control function 261 performs control (hereinafter referred to as “energy control”) to execute scanning in each frame with ultrasonic wave having energy in accordance with the length of a set wait time. For example, when a time accounting for 100% of the required time “Ts” is set as the wait time “Tw”, the wait time is “Tw=Ts” and the inter-frame time interval is “Tf−2×Ts”. In other words, the inter-frame time interval is twice as long as that of a case in which no wait time is set (the case illustrated in FIG. 23). In this case, the control function 261 sets the energy of ultrasonic wave transmitted at scanning in each frame to be twice as large as that of the case in which no wait time is set. Specifically, the control function 261 doubles the sound pressure (drive voltage) of ultrasonic wave transmitted from each transducer element at each scanning. In FIG. 12B, the inter-frame time interval “Tf” is equal to the required time “Ts” (Tf=Ts) because no wait time is set. The set magnification of the energy of ultrasonic wave is not limited to twice as described later.

In this manner, the control function 261 sets the wait time “Tw” during which no scanning is executed, and controls the transmission and reception circuitry 210 such that scanning is executed with ultrasonic wave having energy increased in accordance with the wait time. This allows the control function 261 to achieve improved image quality of an ultrasonic image obtained through each scanning. Accordingly, the ultrasonic diagnostic device 2 can achieve improved visibility of the ultrasonic image.

During the wait time, the control function 261 performs control (hereinafter referred to as “electrical power control”) to reduce electrical power supplied to the electronic circuit included in the ultrasonic probe 201. Specifically, when scanning in the N-th frame ends, the control function 261 stops supply of electrical power to the electronic circuit included in the ultrasonic probe 201. Then, when scanning in the (N+1)-th frame starts, the control function 261 resumes supply of electrical power to the electronic circuit included in the ultrasonic probe 201.

In this manner, the control function 261 completely cuts off the electrical power supplied to the electronic circuit in the ultrasonic probe 201 during the wait time. This allows the control function 261 to achieve a cooling effect by preventing temperature increase during the wait time. This cooling effect is more significant as a larger number of electronic circuits are included in the ultrasonic probe 201 like a 2D array probe, for example.

FIGS. 12A and 12B are merely examples, and the embodiment is not limited to the above description. In the example illustrated in FIG. 12A, the energy of ultrasonic wave is doubled, but embodiments are not limited thereto. In the case illustrated in FIG. 12A, the magnification of the energy of ultrasonic wave can be set to an optional value up to two. Thus, in this case, the magnification of the energy of ultrasonic wave may be to, for example, 1.8.

For example, in FIG. 12A, a time accounting for 100% of the required time “Ts” is set as the wait time “Tw”, but embodiments are not limited thereto. Thus, the control function 261 can optionally set the wait time “Tw” and the energy of ultrasonic wave. For example, when a time accounting for 50% of the required time “Ts” is set as the wait time “Tw”, the control function 261 can set the magnification of the energy of ultrasonic wave to be an optional value up to 1.5. Depending on a value set as the wait time “Tw”, the control function 261 can set the magnification of the energy of ultrasonic wave to be a value larger than two (for example, three or four). For example, when heat generation at the electronic circuit accounts for 90% of heat generation at the entire ultrasonic probe 201 (for example, increase of 9° C. due to heat generation at the electronic circuit), and acoustic heat generation at transducer elements accounts for 10% (for example, increase of 1° C. due to acoustic heat generation) of heat generation at the entire ultrasonic probe 201, doubling its stopping time decreases an increase of the temperature of the electronic circuit from 9° C. to 4.5° C. In this case, the difference of 4.5° C. allows extra acoustic heat generation equivalent to an increase of 4.5° C. from 1° C., that is, transmission at an acoustic power can be 4.5 times larger.

For example, in FIG. 12A, the energy of ultrasonic wave is increased through increase in the sound pressure of ultrasonic wave, but embodiments are not limited thereto. For example, the control function 261 may increase the energy of ultrasonic wave by increasing the frequency of ultrasonic wave and the transmission opening (opening width) of ultrasonic wave other than the sound pressure of ultrasonic wave. In the example illustrated in FIG. 12A, the frequency of ultrasonic wave may be doubled, and the transmission opening may be doubled. The control function 261 may increase the energy of ultrasonic wave by increasing the sound pressure, frequency, and transmission opening of ultrasonic wave in combination as appropriate. In the example illustrated in FIG. 12A, the control function 261 may individually increase the sound pressure, frequency, and transmission opening of ultrasonic wave so as to achieve, with the sum of these increases, the magnification of energy in accordance with the wait time.

For example, in FIG. 12A, increase in the temperature of the electronic circuit is prevented by completely cutting off supply of electrical power to the ultrasonic probe 201 during the wait time “Tw”, but embodiments are not limited thereto. For example, the control function 261 does not necessarily need to completely cut off supply of electrical power to the ultrasonic probe 201. Specifically, the control function 261 may reduce supply of electrical power to the ultrasonic probe 201 so as to reduce heat generation in accordance with reduction in the electrical power.

For example, in FIG. 12A, the B mode scanning is executed as exemplary scanning, but embodiments are not limited thereto. For example, the scanning executed by the transmission and reception circuitry 210 is not limited to the B mode scanning, but may be another kind of scanning such as the color Doppler mode scanning. The scanning executed by the transmission and reception circuitry 210 is not limited to a single kind of scanning, but may be, for example, scanning as a combination of a plurality of kinds of scanning. Examples of the scanning as a combination of a plurality of kinds of scanning include scanning as a combination of the color Doppler mode scanning and the B mode scanning as described in the first embodiment.

For example, in FIG. 12A, the control function 261 executes two controls of the energy control and the electrical power control. These two controls do not necessarily need to be simultaneously executed. In other words, the control function 261 may execute one of the energy control and the electrical power control.

Specifically, when the energy control is to be performed, the ultrasonic diagnostic device 2 includes the transmission and reception circuitry 210, the image processing circuitry 230, and the control function 261. The transmission and reception circuitry 210 repeatedly executes scanning of the subject P. The image processing circuitry 230 generates an ultrasonic image on the basis of reflected wave data collected through the scanning. The control function 261 controls the transmission and reception circuitry 210 so that scanning is executed with ultrasonic wave having energy in accordance with the length of a wait time corresponding to a time from the end of the previous scanning until the start of the next scanning.

When the electrical power control is to be performed, the ultrasonic diagnostic device 2 includes the transmission and reception circuitry 210, the image processing circuitry 230, and the control function 261. The transmission and reception circuitry 210 repeatedly executes scanning of the subject P through the transducer elements. The image processing circuitry 230 generates an ultrasonic image on the basis of reflected wave data collected through the scanning. During a wait time corresponding to a time from the end of the previous scanning until the start of the next scanning, the control function 261 reduces electrical power supplied to the electronic circuit included in the ultrasonic probe 201 including the transducer elements.

### Modification of Second Embodiment

The second embodiment above describes the case in which the control function 261 sets a wait time, but embodiments are not limited thereto. Control by the control function 261 may be executed by using, for example, the wait time Δt of the color Doppler mode scanning described in the first embodiment.

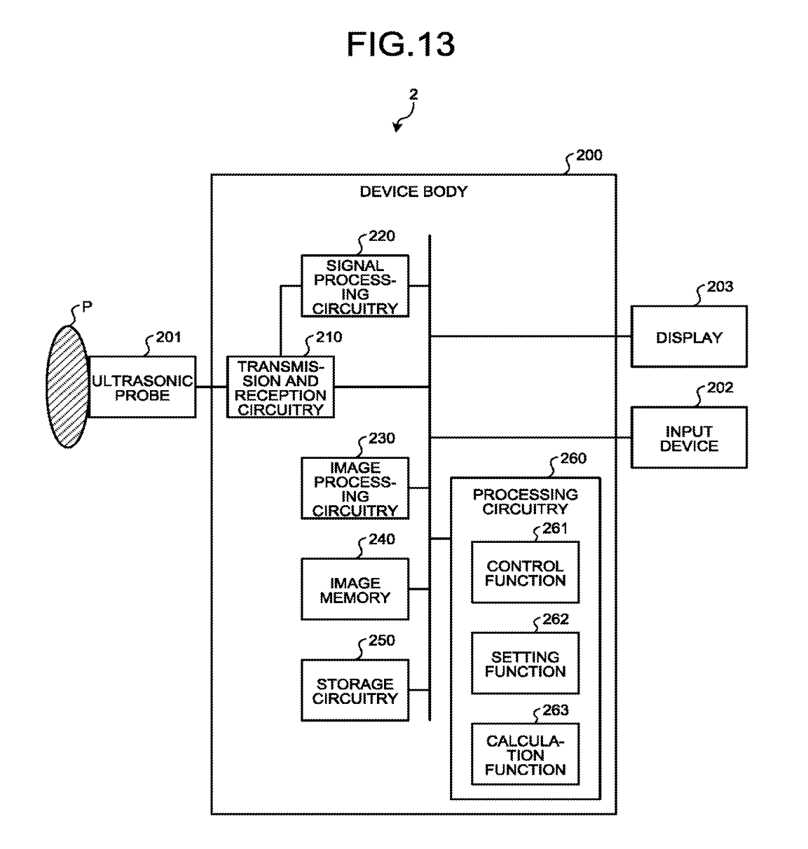

FIG. 13 is a block diagram of an exemplary configuration of the ultrasonic diagnostic device 2 according to a modification of the second embodiment. The ultrasonic diagnostic device 2 according to the modification of the second embodiment has the same configuration as that of the ultrasonic diagnostic device 2 exemplarily illustrated in FIG. 11, but differs therefrom in that the processing circuitry 260 further has a setting function 262 and a calculation function 263. The setting function 262 the calculation function 263 have the same functions as those of the setting function 161 and the calculation function 162 exemplarily illustrated in FIG. 1, respectively, and thus descriptions thereof are omitted.

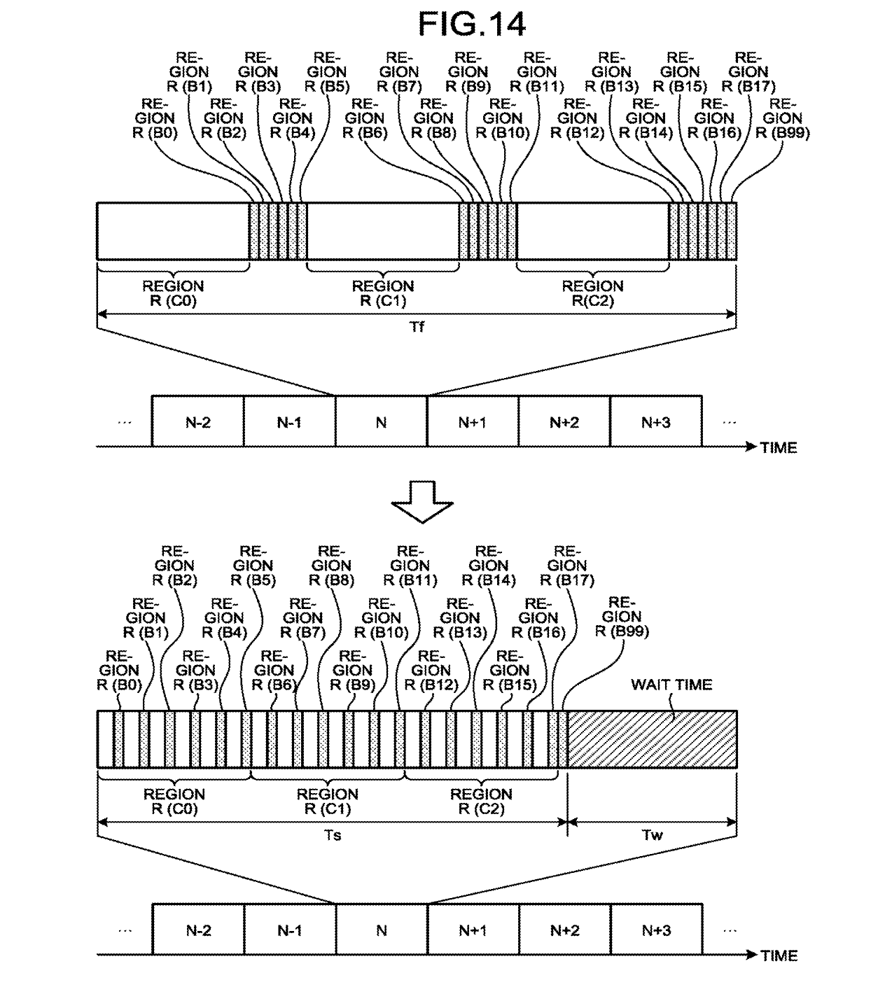

FIG. 14 is a diagram illustrating processing at the control function 261 according to the modification of the second embodiment. FIG. 14 exemplarily illustrates a timing chart when scanning is performed in the regions exemplarily illustrated in FIGS. 3 and 4. The upper section of FIG. 14 exemplarily illustrates a case in which the processing at the ultrasonic diagnostic device 1 according to the first embodiment is not applied. The lower section of FIG. 14 exemplarily illustrates a case in which the processing at the ultrasonic diagnostic device 1 according to the first embodiment is applied. The order of scanning in the lower section of FIG. 14 corresponds to the order of scanning in FIGS. 5 and 6.

As illustrated in the upper section of FIG. 14, when the processing at the ultrasonic diagnostic device 1 according to the first embodiment is not applied, the transmission and reception circuitry 210 executes, in each frame, scanning of the regions illustrated in FIGS. 3 and 4 in an order as follows. Specifically, in each frame, the transmission and reception circuitry 210 executes the color Doppler mode scanning of region R(C0) and then sequentially executes the mode scanning of regions R(B0) to R(B5). Subsequently, the transmission and reception circuitry 210 executes the color Doppler mode scanning of region R(C1) and then sequentially executes the B mode scanning of regions R(B6) to R(B11). Then, the transmission and reception circuitry 210 executes the color Doppler mode scanning of region R(C2) and then sequentially executes the B mode scanning of regions R(B12) to R(B17). Thereafter, the transmission and reception circuitry 210 executes the B mode scanning of region R(B99).

As illustrated in the lower section of FIG. 14, when the processing at the ultrasonic diagnostic device 1 according to the first embodiment is applied, the transmission and reception circuitry 210 executes scanning of the regions in the order illustrated in FIG. 14 in each frame. Specifically, in each frame, the transmission and reception circuitry 210 executes each of the B mode scanning of regions R(B0) to R(B5) during the wait time Δt of the color Doppler mode scanning while executing the color Doppler mode scanning of region R(C0). Subsequently, the transmission and reception circuitry 210 executes each of the B mode scanning of regions R(B6) to R(B11) during the wait time Δt of the color Doppler mode scanning while executing the color Doppler mode scanning of region R(C1). Then, the transmission and reception circuitry 210 executes each of the B mode scanning of regions R(B12) to R(B17) during the wait time Δt of the color Doppler mode scanning while executing the color Doppler mode scanning of region R(C2). Thereafter, the transmission and reception circuitry 210 executes the B mode scanning of region R(B99).

In the lower section of FIG. 14, when the inter-frame time interval “Tf” is set to be equal to the time interval “Tf” in the upper section of FIG. 14, execution of the B mode scanning during the wait time Δt generates the wait time “Tw”.

Then, the control function executes scanning in each frame with ultrasonic wave having energy in accordance with the length of the wait time “Tw” illustrated in FIG. 14. For example, when the wait time “Tw” accounts for 30% of the required time “Ts”, the control function 261 sets the energy of ultrasonic wave transmitted through scanning in each frame to be 1.3 times as large. Accordingly, the control function 261 can achieve improved image quality of an ultrasonic image obtained through each scanning without reduction in the frame rate.

The wait time “Tw” described with reference to FIG. 14 is optionally changeable. For example, the wait time “Tw” illustrated in FIG. 14 may be extended to achieve further improved image quality of an ultrasonic image, or may be shortened to achieve an improved frame rate.

In FIG. 14, the wait time “Tw” is used to perform the energy control, but embodiments are not limited thereto. The control function 261 may execute at least one of the energy control and the electrical power control as described in the second embodiment.

### Other Embodiments

In addition to the embodiments described above, various kinds of different configurations may be applied.

In the above-described embodiments, during the wait time of the color Doppler mode scanning (the first scanning), the B mode scanning (second scanning) as a different kind of scanning is executed, but embodiments are not limited thereto. For example, the transmission and reception circuitry 110 may execute scanning by a pulsed wave Doppler (PWD) method as a different kind of scanning during the wait time of the color Doppler mode scanning. Alternatively, for example, the transmission and reception circuitry 110 may execute the color Doppler mode scanning at different spatial positions during the wait time of the color Doppler mode scanning. Specifically, the transmission and reception circuitry 110 may execute, as the second scanning executed during the wait time of the first scanning, any one of scanning of a kind identical to that of the first scanning but at different spatial positions, scanning of a kind different from that of the first scanning but at identical spatial positions, and scanning of a kind different from that of the first scanning and at different spatial positions.

Components of devices illustrated in each drawing represent functional concepts and do not necessarily need to be physically configured as illustrated in the drawing. In other words, specific distribution and integration of the devices are not limited to those illustrated in the drawing, but all or part thereof may be functionally or physically distributed and integrated in arbitrary units in accordance with various kinds of loads, use conditions, and the like. For example, the processing circuitry 160, 260 may include a processing function of the image processing circuitry 130, 230. In this case, the processing circuitry 160, 260 generates the color Doppler image. In addition, all or an optional part of processing functions performed by the devices may be achieved by a CPU and a computer program analyzed and executed by this CPU, or may be achieved as wired logic hardware.

Among the pieces of processing described in the first embodiment and the modifications thereof, all or some of the pieces of processing described as automatically performed processing may be manually perform, or all or some of the pieces of processing described as manually performed processing may be automatically performed by a well-known method. In addition, information including processing procedures, control procedures, specific names, various kinds of data and parameters described in the above specification and drawings may be optionally changed unless otherwise stated.

The ultrasonic imaging method described in the first embodiment and the modifications thereof may be achieved by a computer such as a personal computer or a work station executing an ultrasonic imaging computer program prepared in advance. This ultrasonic imaging method may be distributed through a network such as the Internet. The ultrasonic imaging method may be recorded on a computer-readable recording medium such as a hard disk, a flexible disk (FD), a CD-ROM, a MO, or a DVD, and read from the recording medium by a computer for execution.

According to at least one of the above-described embodiments, the visibility of an ultrasonic image can be improved.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnostic device comprising:
transmission and reception circuitry configured to repeatedly execute first scanning in which a subject is scanned along a plurality of scanning lines;
processing circuitry configured to control
generating a color Doppler image based on reflected wave data collected through the first scanning; and
obtaining a wait time of the first scanning based on a repetition period of the first scanning and a required time of the first scanning, wherein
the transmission and reception circuitry executes second scanning different from the first scanning during the wait time obtained by the processing circuitry.

2. The ultrasonic diagnostic device according to claim 1, wherein
the repetition period corresponds to a period in which the first scanning is repeated,
the required time corresponds to a minimum necessary time for scanning along the scanning lines, and
the wait time corresponds to a time from end of the previous first scanning until start of the next first scanning.

3. The ultrasonic diagnostic device according to claim 1, wherein the repetition period includes the required time and the wait time.

4. The ultrasonic diagnostic device according to claim 3, wherein the wait time is obtained as a difference between the repetition period and the required time.

5. The ultrasonic diagnostic device according to claim 1, wherein the processing circuitry calculates the wait time by subtracting the required time calculated based on the position of a lower end of a region of interest of the color Doppler image from the repetition period calculated based on a flow speed range of the color Doppler image.

6. The ultrasonic diagnostic device according to claim 1, wherein the processing circuitry:
sets a flow speed range, and
calculates the repetition period based on the set flow speed range.

7. The ultrasonic diagnostic device according to claim 1, wherein the processing circuitry calculates the wait time based on a sound speed, the position of a region of interest the color Doppler image of which is generated, the size of the region of interest, the number of transmission scanning lines arranged in the region of interest, and a reception frequency.

8. The ultrasonic diagnostic device according to claim 1, wherein the transmission and reception circuitry executes, as the first scanning, scanning in which the position of a scanning line is sequentially changed at each transmission and reception of ultrasonic wave.

9. The ultrasonic diagnostic device according to claim 1, wherein the transmission and reception circuitry executes, as the second scanning, any one of scanning of a kind identical to the kind of the first scanning but at different spatial positions, scanning of a kind different from the kind of the first scanning but at identical spatial positions, and scanning of a kind different from the kind of the first scanning and at different spatial positions.

10. The ultrasonic diagnostic device according to claim 1, wherein, when a time taken for the second scanning is shorter than the wait time, the processing circuitry sets, in the wait time, a time during which the second scanning is not executed.

11. The ultrasonic diagnostic device according to claim 1, wherein:
the transmission and reception circuitry executes the first scanning of each of a plurality of regions included in a region of interest, and
the processing circuitry controls generating the color Doppler image corresponding to the region of interest based on reflected wave data collected through the first scanning executed for each of the regions.

12. The ultrasonic diagnostic device according to claim 1, wherein the transmission and reception circuitry executes the first scanning and the second scanning at an identical frame rate.

13. The ultrasonic diagnostic device according to claim 1, wherein
the transmission and reception circuitry executes, as the first scanning and the second scanning, three-dimensional scanning that collects reflected wave data in a three-dimensional space, and
the processing circuitry controls generating three-dimensional image data as the color Doppler image.

14. An ultrasonic diagnostic device comprising:
transmission and reception circuitry configured to repeatedly execute scanning of a subject;
processing circuitry configured to control
generating an ultrasonic image based on reflected wave data collected through the scanning; and
the transmission and reception circuitry so that the scanning is executed with ultrasonic wave having energy in accordance with the length of a wait time corresponding to a time from end of the previous scanning until start of the next scanning.

15. The ultrasonic diagnostic device according to claim 14, wherein the processing circuitry sets, as the wait time, a time during which the scanning is not executed, and executes the scanning with ultrasonic wave having energy in accordance with the length of the set wait time.

16. The ultrasonic diagnostic device according to claim 14, wherein, during the wait time, the processing circuitry reduces electrical power supplied to an electronic circuitry included in an ultrasonic probe performing the scanning.

17. The ultrasonic diagnostic device according to claim 14, wherein the transmission and reception circuitry executes, as the scanning, a single kind of scanning or scanning as a combination of a plurality of kinds of scanning.

18. An ultrasonic diagnostic device comprising:
transmission and reception circuitry configured to repeatedly execute scanning of a subject through a transducer element;
processing circuitry configured to control
generating an ultrasonic image based on reflected wave data collected through the scanning; and
reducing, during a wait time corresponding to a time from end of the previous scanning until start of the next scanning, electrical power supplied to an electronic circuitry included in an ultrasonic probe including the transducer element.

19. The ultrasonic diagnostic device according to claim 18, wherein the processing circuitry sets, as the wait time, a time during which the scanning is not executed, and reduces the electrical power during the set wait time.

20. The ultrasonic diagnostic device according to claim 18, wherein the transmission and reception circuitry executes, as the scanning, a single kind of scanning or scanning as a combination of a plurality of kinds of scanning.
